(19) United States
(12) Patent Application Publication
(10) Pub. No.: US 2003/0022265 A1
(43) Pub. Date: Jan. 30, 2003

(54) Method for determining substrate specificity of protease

(76) Inventors: Woo-Jin Park, Kwangju (KR); Sung-Yun Kim, Kwangju (KR); Dae-Ho Park, Kwangju (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/192,869

(22) Filed: Jul. 8, 2002

(30) Foreign Application Priority Data
Jul. 9, 2001 (KR) 10-2001-0041011

(51) Int. Cl. C12Q 1/68; C12Q 1/37; C12Q 1/04; C12N 1/18; C12N 15/74

(52) U.S. Cl. 435/23; 435/34; 435/254.21; 435/320.1; 435/6

(57) ABSTRACT

The present invention relates to a method for determining substrate specificity of a protease by employing a microorganism cotransformed with a vector expressing the protease and a vector expressing the substrate. The method comprises constructing an expression vector containing a gene for protease; constructing an expression vector containing genes for Golgi recognition signal and transmembrane domain of membrane protein locating at yeast Golgi complex, substrate domain with specific amino acid sequence, and yeast invertase; preparing a transformant by cotransforming suc2 mutant yeast with the two expression vectors; and, determining viability of the transformant in a medium containing a sole carbon source of sucrose. The invented method requires neither isolation/purification of protease nor synthesis of expensive substrate peptide. The simple and cost-effective determination of substrate specificity of proteases can be realized to allow its wide application in the identification of genes for proteases or genes for proteins cleaved with the proteases.

### BACKGROUND OF THE INVENTION

### Field of the Invention

The present invention relates to a method for determining substrate specificity of a protease, more specifically, to a method for determining substrate specificity of a protease by employing a microorganism cotransformed with a vector expressing the protease and a vector expressing the substrate.

Proteases play crucial roles in biochemical actions in vivo, indicating that understanding of their inhibition and activation mechanisms may serve one of the key steps to cure many diseases. In general, researches on protease action are focused on the study of substrate specificity, which is achieved by isolating a protease to be studied, reacting with a substrate peptide containing a specific amino acid sequence, and determining the substrate specificity (amino acid sequence recognized and cleaved by the protease) of the protease.

However, researches on protease action have not been easily made for the following reasons: isolation and purification of proteases, either in nature or already characterized, require a lot of time, effort and cost; and, substrate peptides used for substrate specificity analyses are very expensive, since they contain specific amino acid sequence and occasionally fluorescent or luminescent reporter sequence. Naturally, efforts to simplify the procedures of isolation/purification of protease and improve detection efficiency of reporter are currently being made, which is, however, proven to be less satisfactory in the sense that the substrate peptide is still costly synthesized.

Under the circumstances, there is a continuing need to develop a method for determining substrate specificity of protease in a simple and cost-effective way by solving the problems mentioned above.

### SUMMARY OF THE INVENTION

The present inventors have made an effort to develop a method for determining substrate specificity of a protease, and found that the substrate specificity of a protease can be determined in a simple and cost-effective manner by employing a suc2 mutant yeast cotransformed with a vector expressing a protease and a vector containing genes for Golgi recognition signal and transmembrane domain of integral membrane protein locating at yeast Golgi complex, a protease substrate sequence, and yeast invertase.

A primary object of the invention is, therefore, to provide a method for determining substrate specificity of a protease by employing a microorganism cotransformed with a vector expressing the protease and a vector expressing the substrate.

### BRIEF DESCRIPTION OF THE DRAWINGS

The above and the other objects and features of the present invention will become apparent from the following descriptions given in conjunction with the accompanying drawings, in which:

FIG. 1 is a schematic representation of rationale of a method for determining substrate specificity of a protease of the invention.

FIG. 2 is photographs showing viability of the transformants constructed by an example of the invention.

FIG. 3 is a schematic representation of a method for constructing expression vectors containing various mutated genes of human protease, TMPRSS2.

### DETAILED DESCRIPTION OF THE PREFERRED EMBODIMENT

The method for determining substrate specificity of a protease of the invention comprises the steps of: constructing an expression vector containing a gene for protease; constructing an expression vector containing genes for Golgi recognition signal and transmembrane domain of membrane protein locating at yeast Golgi complex, substrate domain with specific amino acid sequence, and yeast invertase; preparing a transformant by cotransforming suc2 mutant yeast with the said two expression vectors; and, determining viability of the transformant in a medium containing a sole carbon source of sucrose.

The method for determining substrate specificity of protease is further illustrated as follows.

Step 1: Construction of Vector Expressing Protease

A vector containing a gene for protease is constructed: shuttle vectors which are stably expressed both in E. coli and in yeast, and contain yeast promoter such as ADH and CUP, and selective marker gene such as trp, leu, ade and ura are preferably employed as the vector.

Step 2: Construction of Vector Expressing Substrate

A vector containing genes for Golgi recognition signal and transmembrane domain of membrane protein locating at yeast Golgi complex, substrate domain with specific amino acid sequence, and yeast invertase is constructed: the Golgi recognition signal localized in Ste13, a yeast Golgi protein, is preferably employed, which is anchored to Golgi membrane via amino acid sequence of FQFND at N-terminus, and the transmembrane domain consisting of 18 hydrophobic amino acids is preferably used. Yeast invertase, a 532 amino acid protein, is a sucrose hydrolyzing enzyme. Shuttle vectors employed in Step 1 are preferably used therein.

Step 3: Preparation of Transformant

A transformant is prepared by cotransforming suc2 mutant yeast with the said two vectors. The suc2 mutant yeast, a mutant lacking a gene for invertase which hydrolyzes sucrose into glucose and fructose, is not able to grow on a plate containing a sole carbon source of sucrose, while it can grow in a sucrose medium when it is transformed with invertase-expressing vector.

Step 4: Determination of Viability

Viability of transformants is determined in a medium containing a sole carbon source of sucrose, where the medium preferably includes liquid medium or solid medium containing agarose, and the transformant is preferably grown at 25 to 35° C. for 3 to 10 days.

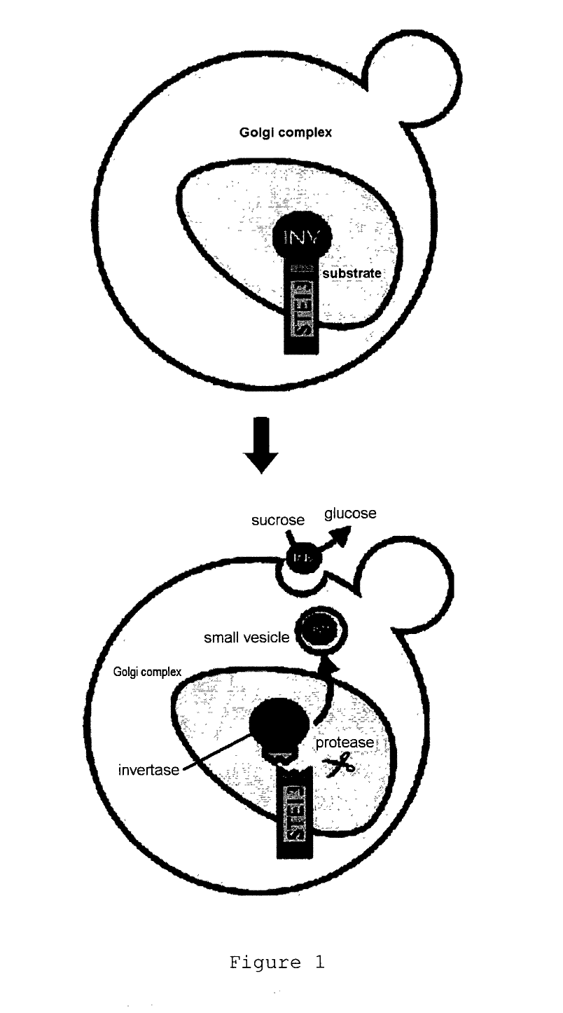

The rationale of the method for determining substrate specificity of a protease is represented in FIG. 1. The present inventors constructed a vector which expresses a protein fused in a way that Golgi recognition signal and transmembrane domain of membrane protein locating at yeast Golgi complex, and substrate domain with specific amino acid sequence were linked in front of yeast invertase. The fusion protein used as a substrate in the invention comprises Golgi recognition signal and transmembrane domain of yeast Golgi protein Ste13, cleavage site of yeast α-factor known as a substrate for Kex2, and yeast invertase. The Golgi recognition signal of Ste13 is located at the 81^{st} amino acid of the fusion protein, the transmembrane domain spans from the 121^{st} to 139^{th} amino acids, and the substrate domain locating at the restriction site between the 161^{st} and 171^{st} amino acids of the fusion protein, exists in the inner side of Golgi complex. Yeast invertase is linked behind substrate insertion site of the fusion protein.

In accordance with an embodiment of the invention, the substrate specificity of a protease can be determined by employing a fusion protein expression vector of the invention. That is, when suc2 mutant yeast is transformed with the said expression vector, the expressed fusion protein is transported to Golgi complex and exists as an integral Golgi membrane protein. Accordingly, invertase in the fusion protein cannot be secreted out of a cell, and the transformant cannot grow on an agar plate containing a sole carbon source of sucrose. When the protease expressed from the protease-expressing vector in the transformant digests the substrate domain of the said fusion protein in Golgi complex, the invertase is separated from the Golgi membrane integrated portion and secreted into the medium to hydrolyze sucrose into glucose and fructose, making the transformant grow on the plate containing sucrose as a sole carbon source. Meanwhile, the transformant cannot grow on the plate containing sucrose as a sole carbon source, when the protease produced from the protease-expressing vector of the transformant cannot digest the substrate domain of the said fusion protein in the Golgi complex. Thus, substrate specificity of a protease can be determined by growing a transformant containing both protease-expressing vector and substrate-expressing vector in a medium containing a sole carbon source of sucrose followed by measuring viability thereof.

In accordance with an embodiment of the invention, a protease can be characterized using the fusion protein expression vector of the invention. That is, a protease of interest can be characterized by employing a vector expressing a fusion protein containing specific substrate for the protease and a vector expressing a protease with specific mutations.

The said transformant was named Saccharomyces cerevisiae SEY6210/pSTE-KR-SUC, pCUP-Kex2, and deposited with the Korean Collection for Type Cultures (KCTC) affiliated to the Korea Research Institute of Bioscience and Biotechnology (KRIBB), an international depository authority, under Accession No. KCTC 1024BP on May 31, 2001.

The invented method for determining substrate specificity of a protease, requires neither isolation/purification of protease nor synthesis of expensive substrate peptide, which makes possible the simple and cost-effective determination of substrate specificity of proteases.

The present invention is further illustrated in the following examples, which should not be taken to limit the scope of the invention.

### EXAMPLE 1

Measurement of Kex2 Activity

A plasmid pADH-kex2 containing kex2 gene linked to ADH promoter was first constructed to express Kex2: pGAD424 vector, which is usually used for yeast two-hybrid experiments, was cut with HindIII to get rid of GAL4 region and then proper restriction sites were inserted to give pADH vector. PCR was performed to amplify kex2 coding sequence using primer 1: 5-CTCGAGATGAAAGTGAGGAAATAT-3 (SEQ ID NO: 1) and primer 2: 5-CTGCAGTCACGATCGTCCGGAAGA-3 (SEQ ID NO: 2), and yeast genomic DNA as a template to obtain 2.4 kb kex2 gene, which was then inserted into XhoI/PstI site of pADH vector to obtain pADH-kex2 expression vector.

The fusion protein used as a substrate comprises Golgi recognition signal and transmembrane domain of yeast Golgi protein Ste13, cleavage site of yeast α-factor known as a substrate for Kex2, and yeast invertase. The recombinant cDNA coding for the said fusion protein was linked to ADH promoter to obtain a plasmid pADH-SteSubInv. More specifically, the substrate-expressing vector pADH-SteSubInv was constructed by replacing GAL4 region of pAS2 vector with genes for invertase and part of Ste13 as described above. Herein, 1.5 kb DNA fragment containing invertase was obtained by PCR amplification using primer 3: 5-ACTAGTATGACAAACGAAACTAGC-3 (SEQ ID NO: 3), primer 4: 5-GACGTCGATAAAATGAAGGGAATG-3 (SEQ ID NO: 4) and yeast genomic DNA as a template, and the DNA fragment containing Golgi recognition signal and transmembrane region of Ste13 was obtained by PCR amplification using primer 5: 5-CTCGAGGTTGTTTTCTTCCAGCCTCATGAC-3 (SEQ ID NO: 5), primer 6: 5-GAATTCTGCCCAAACTAGAATCTCCTGC-3 (SEQ ID NO: 6) and yeast genomic DNA as a template. DNA fragment encoding cleavage site found in α-factor was synthesized based on the amino acid sequence, N-VVMYRREAEA-C (SEQ ID NO: 7).

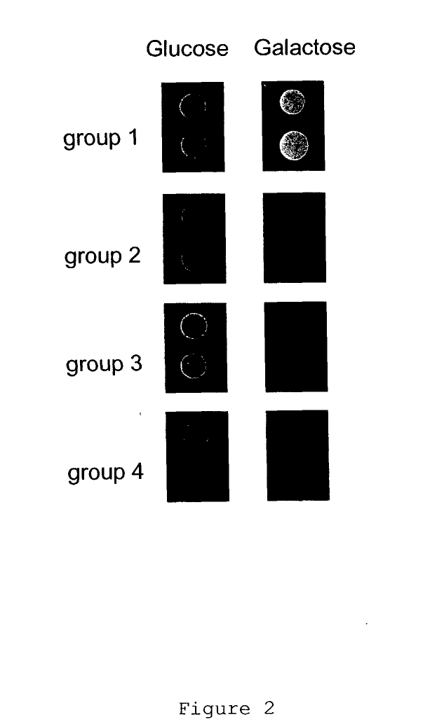

Test group 1 transformants were prepared by cotransforming suc2, kex2 mutant yeast with the said two expression vectors. As control groups, test group 2 transformants were prepared by cotransforming the said yeast with a plasmid pADH which lacks kex2 gene and a plasmid pADH-SteInv which lacks gene for fusion protein, test group 3 transformants were prepared with a plasmid pADH and a plasmid pADH-SteSubInv, and test group 4 transformants were prepared with a plasmid pADH-kex2 and a plasmid pADH-SteInv, respectively. The transformants of test groups 1 to 4 were inoculated onto YPD agar plate (1% (w/v) yeast extract, 2% (w/v) bacto-peptone, 2% (w/v) dextrose, 1% (w/v) agarose) containing 5% (w/v) glucose and the YDP agar plate containing 5% (w/v) sucrose, respectively, and cultured at 30° C. for 7 days (see: FIG. 2). FIG. 2 is a photograph showing viability of the transformants of individual test groups. As shown in FIG. 2, transformants of test group 1 were survived both on glucose-containing and sucrose-containing plates, while, transformants of test groups 2 to 4 were not able to grow on sucrose plates, showing that Kex2 activity can be specifically determined by using the invented method.

The said transformant was named Saccharomyces cerevisiae SEY6210/pSTE-KR-SUC, pCUP-Kex2, and deposited with the Korean Collection for Type Cultures (KCTC) affiliated to the Korea Research Institute of Bioscience and Biotechnology (KRIBB), an international depository authority, under Accession No. KCTC 1024BP on May 31, 2001.

### EXAMPLE 2

Determination of Substrate Specificity

To verify whether the method of the invention can be employed in studying substrate specificity of proteases, substrate specificity of kex2 was assessed by using mutated (amino acids in α-factor region have been substituted) pADH-SteSubInv (constructed in Example 1): a plasmid library was prepared by random substitution of the 5^{th} and 6^{th} amino acids of cleavage region of α-factor, which is the substrate included in pADH-SteSubInv, and then, yeast suc2, kex2 mutant was transformed with pADH-kex2 to obtain primary transformant, followed by transformation with the said plasmid library to obtain secondary transformants. The individual secondary transformants were inoculated onto YPD agar plates containing 5% (w/v) sucrose and incubated at 30° C. for 4 days. The colonies grown were selected to determine nucleotide sequence of α-factor in them. Various amino acids such as D, E, A, V, P, S, Q, K, R, and H were found at the 6^{th} amino acid, while, solely R was found at the 5^{th} amino acid, indicating that survived colonies contain the same amino acid sequences with those found in kex2 substrates. Thus, it was clearly demonstrated that the invented method can be effectively employed in studying the substrate specificity of proteases.

### EXAMPLE 3

Characterization of Proteases

To examine whether the method of the invention can be employed in characterizing proteases, assayed was a human protease TMPRSS2, a 382 amino acid long serine protease, which includes C-terminal catalytic domain of 249 amino acids, LDL receptor domain of 41 amino acids and scavenger receptor domain of 92 amino acids.

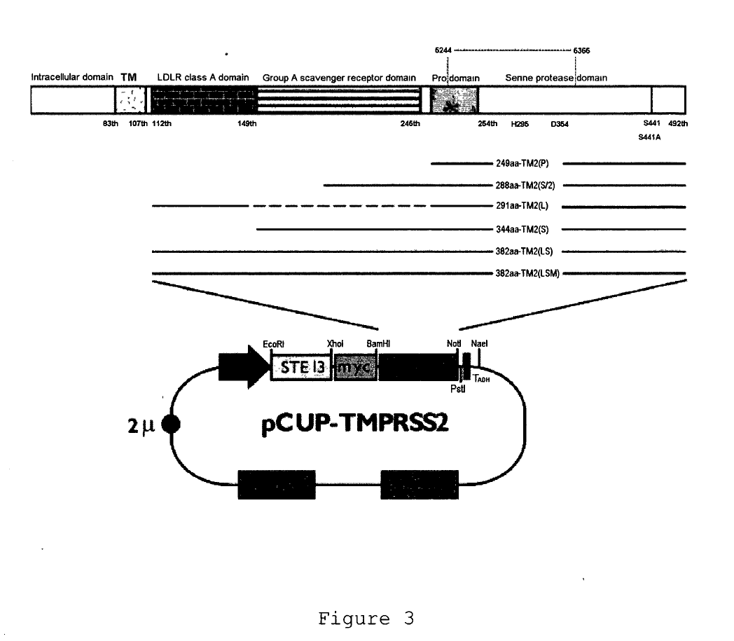

To construct various expression vectors, obtained genes using PCR were LDL receptor domain-deleted mutant gene (344aa-TM2(S)), scavenger receptor domain-deleted mutant gene (291 aa-TM2(L)), a part of scavenger receptor domain-deleted mutant gene (288aa-TM2(S/2)), both LDL receptor domain and scavenger receptor domain-deleted mutant gene (249aa-TM2(P)), normal TMPRSS2 gene (382aa-TM2(LS)), and TMPRSS2 gene with mutation at serine residue in catalytic domain (382aa-TM2(LSM)), which were then linked to 3′-terminal of myc gene, respectively, and STE13 gene was placed in front of myc gene. These constructs were inserted respectively into pCUP expression vector containing ampicillin resistance gene (see: FIG. 3). FIG. 3 is a schematic representation of the method for constructing expression vectors containing various mutations of the said TMPRSS2.

An expression vector producing substrate was constructed in a similar manner as in Example 1, except for inserting a synthetic peptide: VNLNSSRQSRIVGGE (SEQ ID NO: 8), a substrate for TMPRSS2, in place of yeast α-factor cleavage site. Herein, TMPRSS2 cleaves the site between R and I of the said synthetic substrate peptide.

The individual expression vectors containing mutant genes of TMPRSS2 and the substrate expression vector constructed above were cotransformed into yeast, and cultured as described in Example 1. Thus, it was found that only two transformants, containing normal TMPRSS2 gene (382aa-TM2(LS)) and containing LDL receptor domain-deleted mutant gene (344aa-TM2(S)), were survived. Thus, it can be concluded that catalytic domain and scavenger receptor domain are required for proteolytic activity of TMPRSS2, indicating that the function of certain domain of protease can be easily determined by employing the method of the invention.

As clearly illustrated and demonstrated above, the present invention provides a method for determining substrate specificity of a protease by employing a microorganism cotransformed with a vector expressing the protease and a vector expressing the substrate. The invented method for determining substrate specificity requires neither isolation/purification of protease nor synthesis of expensive substrate peptide, by which the simple and cost-effective determination of substrate specificity of proteases can be realized to allow its wide application in the identification of genes for proteases or genes for proteins cleaved with the proteases.

What is claimed is:

1. A plasmid pADH-kex2 containing kex2 gene linked to ADH promoter.

2. A plasmid pADH-SteSubInv containing a recombinant cDNA coding for a fusion protein linked to ADH promoter, wherein the fusion protein comprises Golgi recognition signal and transmembrane domain of yeast Golgi protein Ste13, cleavage site of yeast α-factor as a substrate for Kex2, and yeast invertase.

3. Saccharomyces cerevisiae SEY6210/pSTE-KR-SUC, pCUP-Kex2 (KCTC 1024BP) cotransformed with a plasmid pADH-kex2 containing kex2 gene linked to ADH promoter and the pADH-SteSubInv of claim 2.

4. A method for determining substrate specificity of a protease which comprises the steps of:
(i) constructing an expression vector containing a gene for protease;
(ii) constructing an expression vector containing genes for Golgi recognition signal and transmembrane domain of membrane protein locating at yeast Golgi complex, substrate domain with specific amino acid sequence, and yeast invertase;
(iii) preparing a transformant by cotransforming suc2 mutant yeast with the said two expression vectors; and,
(iv) determining viability of the transformant in a medium containing a sole carbon source of sucrose.

5. The method for determining substrate specificity of a protease of claim 4, wherein the expression vector containing a gene for protease is pADH-kex2.

6. The method for determining substrate specificity of a protease of claim 4, wherein the Golgi recognition signal domain is localized in Ste13.

7. The method for determining substrate specificity of a protease of claim 4, wherein the expression vector containing genes for Golgi recognition signal and transmembrane domain of membrane protein locating at yeast Golgi complex, substrate domain with specific amino acid sequence, and yeast invertase is pADH-SteSubInv.

8. The method for determining substrate specificity of a protease of claim 4, wherein the transformant is Saccharomyces cerevisiae SEY6210/pSTE-KR-SUC, pCUP-Kex2 (KCTC 1024BP).

9. The method for determining substrate specificity of a protease of claim 4, wherein the transformant is grown at 25 to 35° C. for 3 to 10 days.


SEQUENCE LISTING (8 sequences)

SEQ ID NO: 1 - DNA, 24 bp, Artificial Sequence
  Artificial primer 1
ctcgagatgaaagtgaggaaatat

SEQ ID NO: 2 - DNA, 24 bp, Artificial Sequence
  Artificial primer 2
ctgcagtcacgatcgtccggaaga

SEQ ID NO: 3 - DNA, 24 bp, Artificial Sequence
  Artificial primer 3
actagtatgacaaacgaaactagc

SEQ ID NO: 4 - DNA, 24 bp, Artificial Sequence
  Artificial primer 4
gacgtcgataaaatgaagggaatg

SEQ ID NO: 5 - DNA, 30 bp, Artificial Sequence
  Artificial primer 5
ctcgaggttgttttcttccagcctcatgac

SEQ ID NO: 6 - DNA, 28 bp, Artificial Sequence
  Artificial primer 6
gaattctgcccaaactagaatctcctgc

SEQ ID NO: 7 - protein, 10 aa, Artificial Sequence
  Artificial Peptide substrate for kex2
VVMYRREAEA

SEQ ID NO: 8 - protein, 15 aa, Artificial Sequence
  Artificial peptide substrate for TMPRSS2
VNLNSSRQSRIVGGE